(19) United States
(12) Patent Application Publication
(10) Pub. No.: US 2017/0360891 A1
(43) Pub. Date: Dec. 21, 2017

(54) Stable, Benzyl Alcohol-free Aqueous Solution Formulations Containing Alpha-type Interferon

(71) Applicant: Hoffmann-La Roche Inc., Little Falls, NJ (US)

(72) Inventors: Ulla Grauschopf, Riehen (CH); Michael Adler, Loerrach (DE); Hanns-Christian Mahler, Loerrach (DE); Felix Heise, Basel (CH); Susanne Baroth, Binningen (CH)

(73) Assignee: Hoffmann-La Roche Inc., Little Falls, NJ (US)

(21) Appl. No.: 15/467,884

(22) Filed: Mar. 23, 2017

Related U.S. Application Data
(63) Continuation of application No. PCT/EP2015/071536, filed on Sep. 21, 2015.

(30) Foreign Application Priority Data
Sep. 23, 2014 (EP) 14186040.3

(51) Int. Cl. A61K 38/21; A61K 47/10; A61K 9/00; A61K 9/08; A61K 47/26; A61K 47/18

(52) U.S. Cl. CPC A61K 38/212; A61K 47/26; A61K 47/183; A61K 9/0019; A61K 9/08; A61K 47/10

(57) ABSTRACT

The present invention provides a stable, isotonic, aqueous solution formulation comprising: (i) 0.1-0.5 mg/mL alpha-type interferon, preferably pegylated alpha-type interferon; (ii) 20 mM Acetate buffer system to maintain a pH of 6.0±0.5; (iii) 5-20 mM L-methionine; (iv) 120-150 mM sodium chloride; (v) 0.01-0.07 percent by weight of a surfactant effective to stabilize the alpha-type interferon, preferably pegylated alpha-type interferon against loss of its activity and (vi) an amount of water for injection sufficient to prepare a solution of the above-listed ingredients.

### CROSS REFERENCE TO RELATED APPLICATIONS

This application is a continuation of International Patent Application No. PCT/EP2015/071536 having an international filing date of Sep. 21, 2015, the entire contents of which are incorporated herein by reference, and which claims benefit under 35 U.S.C. 119 to European Patent Application No. 14186040.3 filed on Sep. 23, 2014.

### FIELD OF THE INVENTION

Stable, benzyl alcohol-free aqueous solution formulations containing alpha-type interferon, preferably pegylated alpha-type interferon, a buffer to maintain a pH of 6.0±0.5, polysorbate 20 or poloxamer 188 as a surfactant, L-methionine as stabilizer, and a tonicity agent and which maintain high chemical and physical stability of the pegylated alpha-type interferon for an extended storage period of time (at the minimum, 18 months at a target storage temperature of 2-8° C. or 6 months at an elevated temperature of 25° C.) are disclosed.

### BACKGROUND OF THE INVENTION

This invention relates to stable, aqueous solution formulations which maintain high biological activity and high chemical and high physical stability of interferon alpha, preferably pegylated interferon alpha for an extended period of time, i.e. at least 18 months, preferably 24 months or more, at the intended storage temperature (2-8° C.) as typically required for a commercial biopharmaceutical product. Physical stability is demonstrated by control of the formation of soluble (oligomeric forms and higher, soluble aggregates as shown e.g. by size exclusion chromatography) and insoluble (visible and/or subvisible particulates) aggregated species, by control of visual aspects like turbidity and discoloration, as well as a constant overall concentration of the active ingredient (shown e.g. by reversed phase HPLC). Chemical stability is demonstrated by control of the degradation of the active ingredient which typically occurs by fragmentation (shown e.g. by reversed phase HPLC), oxidation (in particular of methionine side chains), deamidation of asparagine residues, isomerization of aspartic acid residues (the latter three shown e.g. by peptide mapping using mass spectrometric analysis of peptides after controlled limited proteolysis).

The manufacture of interferon solutions involves a number of problems which are caused by the sensitivity of the active ingredient against physical and chemical influences. Like other proteins interferon in aqueous solutions is subject to chemical degradation mechanisms such as proteolysis, oxidation, disulfide exchange, oligomerization, deamidation and beta-elimination, and physical mechanisms such as aggregation, precipitation and adsorption. Interferon solutions therefore contain additives which are present to counteract these effects.

U.S. Pat. No. 4,496,537 discloses biologically stable interferon alpha aqueous solution formulations containing interferon alpha, human serum albumin and alanine or glycine, water, and a buffer system to maintain the pH at 6.5-8.0. The human serum albumin (“HSA”) acts as a stabilizer for interferon alpha and prevents losses of interferon alpha from solution by coating and/or adsorption of the interferon alpha onto the stainless steel and glass surfaces of compounding vessels, process equipment and storage containers. Solution formulations containing interferon alpha and HSA have maintained the chemical and biological stability of the interferon alpha when such solutions have been stored at 2-8° C. for extended periods, i.e., more than 2 years. However, HSA has been problematic in view of potential viral contamination and formation of covalent aggregates (via disulphide formation/disulphide shuffling with its free thiol groups) which in turn may cause immunogenicity potentially leading to loss of efficacy or even anaphylactic reactions.

Later, interferon alpha solutions have been proposed which avoid the use of HSA and which contain other auxiliary agents, inter alia, non-ionic detergents (see for instance WO 89/04177). Since interferon alpha is highly active and is present in minimal concentration in pharmaceutical preparations, the stability of interferon preparations and ensuring a constant concentration of the active ingredient is of particular importance. It has been found that in order to guarantee optimal utilization properties the excipients of an interferon solution must be selected carefully from a multitude of potentially suitable agents and be harmonized with each other. For example, the adsorption of interferon-alpha 2a on glass surfaces has a maximum at pH 5-6 so that this pH would in principle seem unfavorable. On the other hand, covalent degradation reactions proceed through a minimum at this pH. Commercial HSA-stabilized solutions have pH 7. The utilization properties of interferon solutions are influenced by a number of non-correlating factors in an unpredictable manner.

U.S. Pat. No. 5,762,923 discloses aqueous HSA-free interferon-alpha solution formulations containing an interferon alpha, a non-ionic detergent, a buffer for adjusting pH 4.5-6.0, benzyl alcohol and, optionally, an isotonizing agent which exhibit optimal utilization properties, i.e. storage stability and bioavailability of the declared amount of active ingredient. More specifically, U.S. Pat. No. 5,762,923 discloses an aqueous interferon solution formulation which contains a pegylated interferon alpha-2a of formula (I),

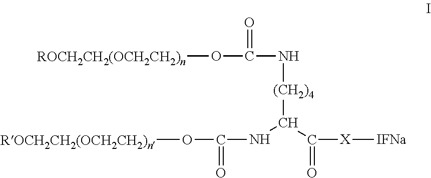

benzyl alcohol of an amount of 10 mg/ml, sodium acetate/acetic acid as buffer adjusted to a final pH 6.0, sodium chloride as tonicity agent and polysorbate 80 as surfactant. It is evident from the data shown in U.S. Pat. No. 5,762,923 that an acceptable storage stability of solutions is achieved, provided the solutions were prepared with the addition of benzyl alcohol.

However, in numerous pharmaceutical and biotechnological applications like the manufacturing process for preparing (pegylated) alpha-type interferon, maintaining fluid integrity (i.e. qualitative and quantitative composition as well as overall volume) during transfer of fluids is critical. Loss of fluid or of one or several of its components e.g. through migration into the tubing walls can cause inconsistencies in final product results. Benzyl alcohol is known to be absorbed by various tubing materials and in particular by tubes made of silicone rubber, which are widely used in many pharmaceutical process facilities.

Therefore, it would desirable to avoid benzyl alcohol from a manufacturing process perspective. However, the benzyl alcohol free alpha-type interferon, preferably pegylated alpha-type interferon formulations should still provide for at least the same storage stability. Hence, there's a need to reformulate existing alpha-type interferon, preferably pegylated alpha-type interferon solution products to obtain a solution formulation free of benzyl alcohol while maintaining high chemical, high physical stability and high interferon alpha, preferably pegylated interferon alpha activity in the aqueous solution formulations for extended storage periods.

### SUMMARY OF THE INVENTION

The present invention is based on the surprising finding that benzyl alcohol previously used as stabilizer in formulations of alpha-type interferons, preferably pegylated alpha-type interferons can be avoided by substituted by L-methionine provided the formulation further comprises polysorbate 20 or poloxamer 188 as surfactant. For instance, replacing merely benzyl alcohol with L-methionine in the commercial formulation of Pegasys® (40 kDa branched pegylated interferon alpha 2a) will result in lower chemical, physical stability and pegylated interferon alpha activity in the aqueous solution formulations for extended storage periods as provided by the commercial Pegasys® formulation. However, the combination of L-methionine with either polysorbate 20 or poloxamer 188 will lead to a formation with a comparable stability.

Without the intention to be bound by any theory, the surfactant in the formulation according to the present invention appears to be more stable because PS20 is less prone to degradation than PS80 which likely leads to a reduced oxidative stress in the resulting formulation. Since L-Methionine does not form peroxides (in contrast to benzyl alcohol), the formulation according to the present invention can be stored without inert gas (e.g. nitrogen) overlay and hence it has an improved storage stability. Further, the drug product manufacturing process is simplified because L-Methionine (in contrast to benzyl alcohol) is not readily absorbed by or adsorbed to plastics or elastomers which are typically used in the manufacturing process (like e.g. silicone tubing, PTFE tubing, plastic connectors) and it does neither evaporate. Thus, line stoppages are less critical and flush volumes or the number of drug product units to be discarded after a line stoppage can be reduced. The formulation according to the present invention also provides for a better stability of the drug product composition because L-Methionine does not evaporate or permeate through elastomeric container closures (like e.g. rubber plunger stoppers) and is also not absorbed by or adsorbed to them.

The present invention provides a stable, isotonic, aqueous solution formulation which maintains high biological alpha-type interferon activity and is free of benzyl alcohol, which comprises:
(i) 0.1-0.5 mg/mL, preferably 0.18, 0.27 or 0.36 mg/mL alpha-type interferon, preferably pegylated alpha-type interferon;
(ii) 20 mM Acetate, preferably sodium acetate buffer system to maintain a pH of 6.0±0.5;
(iii) 5-20 mM, preferably 10 mM L-methionine;
(iv) 120-150 mM, preferably 130-140 mM or 137 mM sodium chloride;
(v) 0.01-0.07, preferably 0.02 percent by weight of a surfactant effective to stabilize the alpha-type interferon, preferably pegylated alpha-type interferon against loss of its activity; and
(vi) an amount of water for injection sufficient to prepare a solution of the above-listed ingredients.

In an alternative embodiment of the present invention, the surfactant is either polysorbate 20 or poloxamer 188. In one embodiment, the surfactant is polysorbate 20.

In a further embodiment of the present invention, the pegylated alpha-type interferon is a physiologically active pegylated alpha-type interferon conjugate of formula (I)

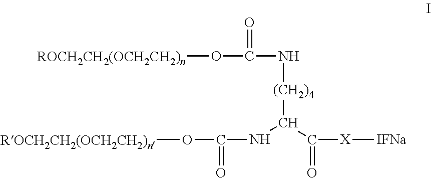

wherein R and R′ are methyl, X is NH, the average sum of n and n′ is 850 to 1000 and the molecular weight of the polyethylene glycol units is about 40 kDa.

In yet another embodiment the above pegylated alpha-type interferon is an alpha-2a interferon.

The present invention further provides a stable aqueous solution formulation comprising:

| (i) | Pegylated alpha-type interferon of formula (I) | 0.18 | mg/mL |
|---|---|---|---|
| | 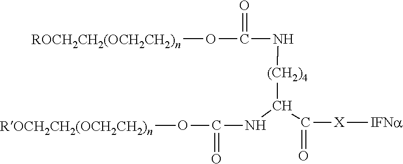 | I | |
| | wherein R and R′ are methyl, X is NH, the average sum of n and | | |
| | n′ is 850 to 1000, the molecular weight of the polyethylene glycol | | |
| | units is about 40 kDa and the IFN alpha is an IFN alpha-2a | | |
| (ii) | Acetate, preferably sodium acetate buffer to maintain a pH of 6.0 ± 0.5 | 20 | mM |
| (iii) | L-methionine | 10 | mM |
| (iv) | Sodium chloride | 137 | mM |

| | | |
|---|---|---|
| (v) | Polysorbate 20 | 0.02 weight-% |
| (vi) | Water for injection | q.s. ad 1 mL. |

The present invention also provides a stable aqueous solution formulation comprising:

| (i) | Pegylated alpha-type interferon of formula (I) | 0.18 | mg/mL |
|---|---|---|---|
| | 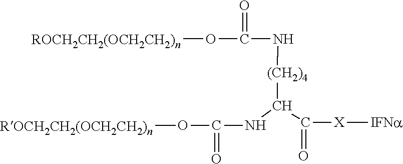 | I | |
| | wherein R and R′ are methyl, X is NH, the average sum of n and | | |
| | n′ is 850 to 1000, the molecular weight of the polyethylene glycol | | |
| | units is about 40 kDa and the IFN alpha is an IFN alpha-2a | | |
| (ii) | Acetate, preferably sodium acetate buffer to maintain a pH of 6.0 ± 0.5 | 20 | mM |
| (iii) | L-methionine | 10 | mM |
| (iv) | Sodium chloride | 137 | mM |

| | | |
|---|---|---|
| (v) | Poloxamer 188 | 0.02 weight-% |
| (vi) | Water for injection | q.s. ad 1 mL. |

The present invention provides yet another stable aqueous solution formulation comprising:

| (i) | Pegylated alpha-type interferon of formula (I) | 0.27 | mg/mL |
|---|---|---|---|
| | 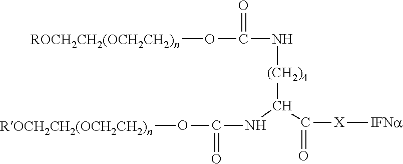 | I | |
| | wherein R and R′ are methyl, X is NH, the average sum of n and | | |
| | n′ is 850 to 1000, the molecular weight of the polyethylene glycol | | |
| | units is about 40 kDa and the IFN alpha is an IFN alpha-2a | | |
| (ii) | Acetate, preferably sodium acetate buffer to maintain a pH of 6.0 ± 0.5 | 20 | mM |
| (iii) | L-methionine | 10 | mM |
| (iv) | Sodium chloride | 137 | mM |

| | | |
|---|---|---|
| (v) | Polysorbate 20 | 0.02 weight-% |
| (vi) | Water for injection | q.s. ad 1 mL. |

The present invention provides yet another stable aqueous solution formulation comprising:

| (i) | Pegylated alpha-type interferon of formula (I) | 0.27 | mg/mL |
|---|---|---|---|
| | 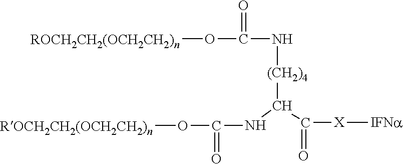 | I | |
| | wherein R and R′ are methyl, X is NH, the average sum of n and | | |
| | n′ is 850 to 1000, the molecular weight of the polyethylene glycol | | |
| | units is about 40 kDa and the IFN alpha is an IFN alpha-2a | | |
| (ii) | Acetate, preferably sodium acetate buffer to maintain a pH of 6.0 ± 0.5 | 20 | mM |
| (iii) | L-methionine | 10 | mM |
| (iv) | Sodium chloride | 137 | mM |

| | | |
|---|---|---|
| (v) | Poloxamer 188 | 0.02 weight-% |
| (vi) | Water for injection | q.s. ad 1 mL. |

The present invention provides yet another stable aqueous solution formulation comprising:

| (i) | Pegylated alpha-type interferon of formula (I) | 0.36 | mg/mL |
|---|---|---|---|
| | 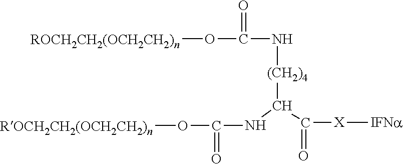 | I | |
| | wherein R and R′ are methyl, X is NH, the average sum of n and | | |
| | n′ is 850 to 1000, the molecular weight of the polyethylene glycol | | |
| | units is about 40 kDa and the IFN alpha is an IFN alpha-2a | | |
| (ii) | Acetate, sodium acetate buffer to maintain a pH of 6.0 ± 0.5 | 20 | mM |
| (iii) | L-methionine | 10 | mM |
| (iv) | Sodium chloride | 137 | mM |

| | | |
|---|---|---|
| (v) | Polysorbate 20 | 0.02 weight-% |
| (vi) | Water for injection | q.s. ad 1 mL. |

The present invention provides yet another stable aqueous solution formulation comprising:

| (i) | Pegylated alpha-type interferon of formula (I) | 0.36 | mg/mL |
|---|---|---|---|
| | 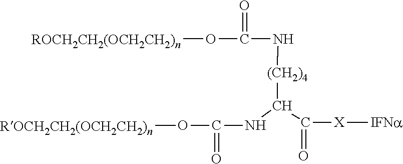 | I | |
| | wherein R and R′ are methyl, X is NH, the average sum of n and | | |
| | n′ is 850 to 1000, the molecular weight of the polyethylene glycol | | |
| | units is about 40 kDa and the IFN alpha is an IFN alpha-2a | | |
| (ii) | Acetate, preferably sodium acetate buffer to maintain a pH of 6.0 ± 0.5 | 20 | mM |
| (iii) | L-methionine | 10 | mM |
| (iv) | Sodium chloride | 137 | mM |

| | | |
|---|---|---|
| (v) | Poloxamer 188 | 0.02 weight-% |
| (vi) | Water for injection | q.s. ad 1 mL. |

### BRIEF DESCRIPTION OF THE DRAWINGS

FIG. 1 shows the ratio of the purity of pegylated alpha-2α interferon to monomer content, as measured by size exclusion chromatography, in different formulations over time at 5° C. storage.

FIG. 2 shows the ratio of the purity of pegylated alpha-2α interferon to monomer content, as measured by size exclusion chromatography, in different formulations over time at 25° C. storage.

FIG. 3 shows the ratio of the purity of pegylated alpha-2α interferon to content of non-oxidized active pharmaceutical ingredient (API), as measured by HPLC, in different formulations over time at 5° C. storage.

FIG. 4 shows the ratio of the purity of pegylated alpha-2α interferon to content of non-oxidized active pharmaceutical ingredient (API), as measured by HPLC, in different formulations over time at 25° C. storage.

### DETAILED DESCRIPTION OF THE INVENTION

We have selected specific amounts of a specific set of ingredients that have allowed us to develop an aqueous pegylated alpha-type interferon solution formulation which does not contain benzyl alcohol yet maintains high chemical, biological and physical stability for the pegylated alpha-type interferon for extended periods of time.

The term “free of benzyl alcohol” or “benzyl alcohol free” as used herein in reference to the formulations of the present invention means that no benzyl alcohol is used in the preparation of the solution formulations of the present invention.

The buffer systems suitable for the formulations of the present invention are those which maintain the Ph of the aqueous solution formulation in the range of 5.5 to 6.5, preferably 5.8-6.2 and most preferably 6.0. The use of a buffer system of sodium acetate/acetic acid is preferred. Other suitable buffer systems to maintain the desired Ph range of 5.5 to 6.5 include sodium citrate/citric acid and sodium phosphate dibasic and sodium phosphate monobasic. The tonicity agent useful in the present invention is any agent capable of rendering the formulations of the present invention iso-osmotic with human serum. Typical suitable tonicity agents include sodium chloride, mannitol, glycine, glucose and sorbitol. Use of sodium chloride as a tonicity agent is preferred.

The sorbitan mono-9-octadecenoate poly(oxy-1,2-ethanediyl) derivative polysorbate 20 is useful as a surfactant to prevent adsorption of the pegylated alpha-type interferon proteins such as 40 kDa branched pegylated alpha-2a interferon (Pegasys®) onto the stainless steel and glass surfaces of the equipment used to make the indictable formulations containing pegylated alpha-type interferon. The amount of polysorbate 20 is in the range of 0.005 to 0.5 percent by weight, preferably 0.02 percent by weight for a formulation containing 0.1 0.5 mg/Ml pegylated alpha-type interferon. Surprisingly, we have found that polysorbate 20 prevents loss of pegylated alpha-2a interferon and allows systemic delivery of the pegylated alpha-2a interferon without loss of biological activity. In the course of development of the formulation of the present invention, we surprisingly found that polysorbate 20 provided superior chemical and biological stability to a pegylated alpha-2a interferon in the presence of L-methionine (replacing benzyl alcohol) compared to other sorbitan mono-9-octadecenoate poly(oxy-1,2-ethanediyl) derivative surfactants, e.g., polysorbate 80.

Similar chemical and biological stability is achieved when poloxamer 188 is used instead of polysorbate 20 at the same concentration.

The amount of pegylated alpha-type interferon useful in the formulation of the present invention is in the range of 0.1 to 0.5 mg/Ml. As used herein the term “pegylated alpha-type interferon” means covalent conjugates of one or more polyethylene glycol (PEG) molecules and one or more alpha-type interferon molecules. Preferred conjugates for use in the formulations of the invention have one to four PEG molecules per interferon molecule, and more preferably, the conjugates are between a single PEG molecule and a single interferon molecule. The pegylated interferon may comprise a single positional isomer or a mixture of conjugate positional isomers, e.g, the PEG molecules are covalently attached to different amino acid residues on the individual interferon molecules. For example, U.S. Pat. No. 5,951,974 describes the preparation of mixtures of PEG-interferon alpha conjugate positional isomers in which some of the isomers are conjugates between PEG and a histidine residue of the interferon molecule, other isomers in the mixture are conjugates between PEG and an interferon lysine residue and still other isomers are conjugates between PEG and the amino terminus of the interferon molecule.

The PEG molecules in the conjugates may have different molecular weights. Preferably, the PEG molecule has an average molecular weight of 40,000. In a particularly preferred embodiment, the conjugates are prepared using a branched PEG₄₀₀₀₀, i.e., which means the PEG molecules in the conjugates will have an average molecular weight of about 40,000.

The interferon portion of the pegylated alpha-type interferon conjugates used in the present invention may be any naturally-occurring or recombinant interferon alpha known to those skilled in the art. Natural and recombinant alpha-interferons that may be used in the formulations of the invention include interferon alpha-n1 (e.g., Surniferon®, Surnitomo®), interferon alpha-n3, interferon alpha-2a (Roferon® A, Hoffmann-LaRoche, Inc.) interferon α-2b (INTRON® A, Schering-Plough Corp.), interferon alpha-2c (Berofor®, Boehringer Ingelheim, Inc.), and consensus interferon (Infergen®, InterMune, Inc.). Preferred interferons are interferon alpha-2a and interferon alpha-2b. Most preferably, interferon alpha-2a is used to prepare the active ingredient of the formulations of the present invention.

Conjugation of the PEG and interferon molecules may be performed by any conjugation reaction known to those skilled in the art, e.g., as described in U.S. Pat. Nos. 5,612,460, 5,711,944 and 5,951,974. Preferably, the PEG molecule is covalently attached to the interferon molecule with a urethane bond.

The most preferred pegylated alpha-type interferon for use in the formulations of the invention is a branched PEG₄₀₀₀₀-interferon alpha-2a.

The water used for preparation of the formulations of the present invention is preferably water for injection.

During the course of development of the aqueous solution formulations of the present invention that would maintain high biological activity as well as high chemical and high physical stability of the pegylated alpha-type interferon over an extended storage period without employing benzyl alcohol as a stabilizer, we identified that L-methionine can only successfully replace benzyl alcohol as stabilizer when either polysorbate 20 or poloxamer 188 is used as surfactant.

Pegylated alpha-type interferon formulations are useful for treatment of a variety of disease states such as renal cell carcinomas, AIDS-related Kaposi's sarcoma, chronic and acute hepatitis B, chronic and acute non-A, non-B/C hepatitis. The formulations of the present invention are useful in treating these disease states preferably as injectable aqueous solutions.

### Examples

The following non-limiting examples illustrate the preparation of the aqueous solutions of pegylated alpha-type interferons.

### Formulation Manufacturing and Composition

Commercial Pegasys drug substance (1-2 mg/Ml Peginterferon alpha-2a, 20 Mm acetic acid/sodium acetate Ph 6.0, 50 Mm sodium chloride) was spiked with different concentrated excipient stock solutions and at the same time diluted in order to yield final drug product formulations containing 0.27 mg/Ml Peginterferon alpha-2a, 20 Mm acetic acid/sodium acetate Ph 6.0, 137 Mm NaCl, L-Methionine at levels indicated below, 0.2 mg/Ml Polysorbate 20 or Poloxamer 188. As a control, the current Pegasys drug product market formulation (0.27 mg/Ml Peginterferon alpha-2a, 20 Mm acetic acid/sodium acetate Ph 6.0, 137 Mm NaCl, 10 mg/Ml benzyl alcohol, 0.05 mg/Ml Polysorbate 80) was compounded following the same procedure. After careful homogenization by stirring, all final bulk solutions were sterile filtered using 0.22 μm hydrophilic PVDF filters. For stability assessment the solutions were aseptically filled into sterile, pre-siliconized glass syringes (fill volume: 1 Ml) and closed with sterile rubber stoppers. The samples were stored at 5° C. and 25° C., respectively, and analyzed for purity at the time points indicated below using analytical procedures established for the commercial drug product (Size Exclusion Chromatography and Reversed Phase HPLC).

### Stability Data

The analytical methods applied to show the purity of the formulations during storage at different temperatures reveal very similar to almost identical stability properties of the new formulations (F1 and F2) compared to the current benzyl alcohol-containing formulation (F20). F17 which contains neither L-methionine nor benzyl alcohol demonstrates that the presence of an agent with antioxidant properties is required to protect the API from oxidation.

F1: Polysorbate 20 containing formulation with 10 Mm L-Methionine, F2: Poloxamer 188 containing formulation with 10 Mm L-Methionine, F17: like F2 but without L-Methionine, F20: current formulation (20 Mm Acetic Acid/Na-Acetate Ph 6.0, 137 Mm NaCl, 10 mg/Ml Benzyl Alcohol, 0.05 mg/Ml Polysorbate 80)

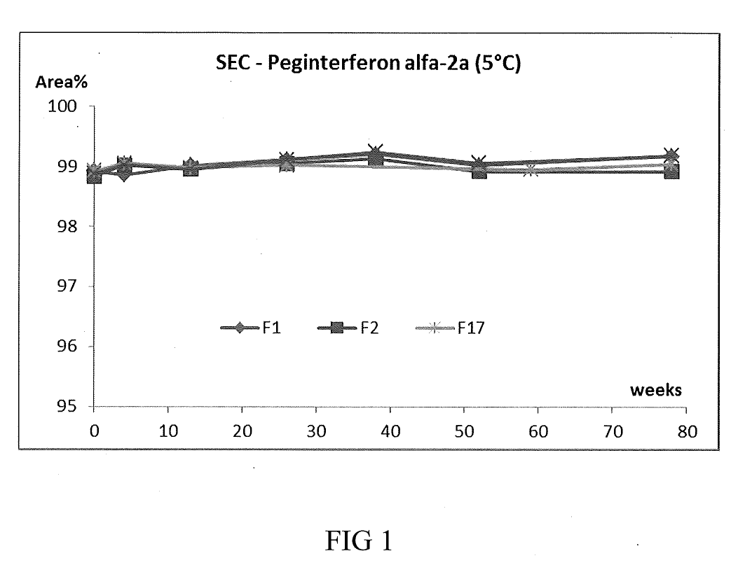

Table 1 below presents data showing the ratio of the purity of pegylated alpha-2α interferon to monomer content, as measured by size exclusion chromatography, in different formulations over time at 5° C. storage. This data is represented in FIG. 1.

TABLE 1

| | | 4 | 13 | 26 | 38 | 52 | 59 | 78 |
|---|---|---|---|---|---|---|---|---|
| Formulation | Initial | weeks | weeks | weeks | weeks | weeks | weeks | weeks |
| 1 | 99.0 | 98.9 | 99.0 | 99.1 | 99.2 | 99.0 | n.t. | 99.2 |
| 2 | 99.0 | 99.1 | 99.0 | 99.1 | 99.2 | 98.9 | n.t. | 98.9 |
| 17 | 99.0 | 99.1 | 98.9 | 99.0 | n.t. | n.t. | 99.0 | 99.0 |
| 20 | 98.9 | 99.0 | 99.0 | 99.1 | 99.2 | 99.1 | n.t. | 99.2 |

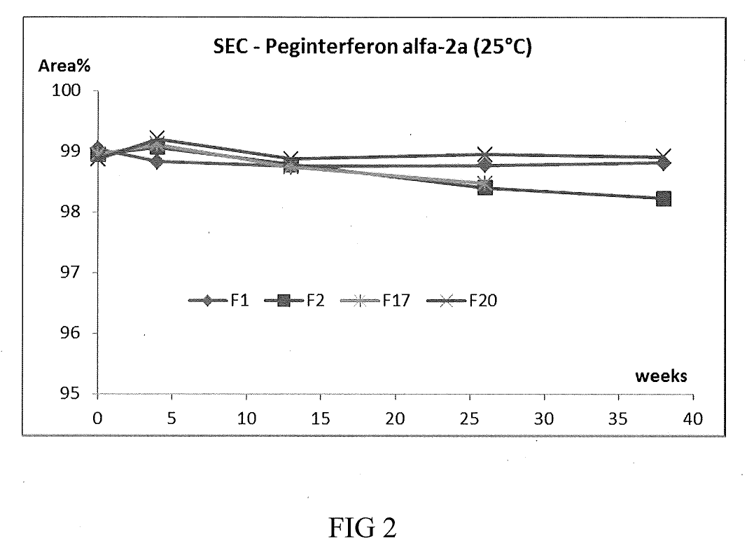

Table 2 below presents data showing the ratio of the purity of pegylated alpha-2α interferon to monomer content, as measured by size exclusion chromatography, in different formulations over time at 25° C. storage. This data is represented in FIG. 2.

TABLE 2

| Formulation | Initial | 4 weeks | 13 weeks | 26 weeks | 38 weeks |
|---|---|---|---|---|---|
| 1 | 99.0 | 98.8 | 98.8 | 98.8 | 98.8 |
| 2 | 99.0 | 99.1 | 98.8 | 98.4 | 98.2 |
| 17 | 99.0 | 99.1 | 98.8 | 98.5 | n.t. |
| 20 | 98.9 | 99.2 | 98.9 | 99.0 | 98.9 |

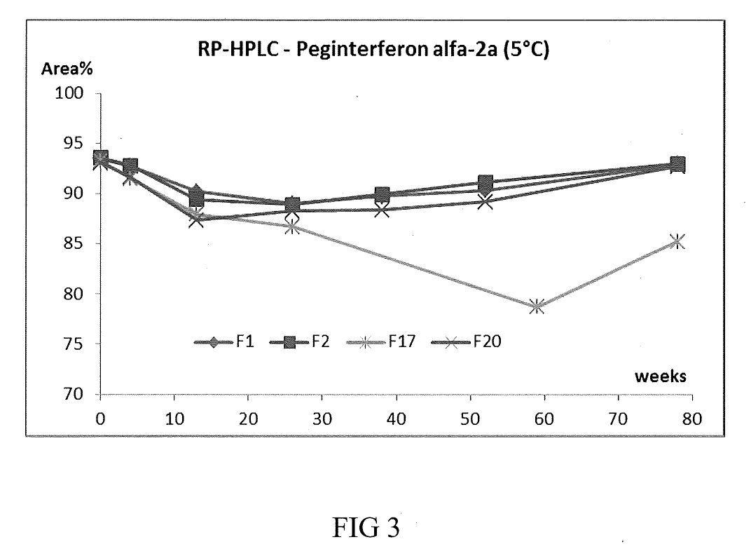

Table 3 below presents data showing the ratio of the purity of pegylated alpha-2α interferon to content of non-oxidized active pharmaceutical ingredient (API), as measured by HPLC, in different formulations over time at 5° C. storage. This data is represented in FIG. 3.

TABLE 3

| | | 4 | 13 | 26 | 38 | 52 | 59 | 78 |
|---|---|---|---|---|---|---|---|---|
| Formulation | Initial | weeks | weeks | weeks | weeks | weeks | weeks | weeks |
| 1 | 93.5 | 92.8 | 90.2 | 89.0 | 89.8 | 90.4 | n.t. | 93.0 |
| 2 | 93.6 | 92.8 | 89.4 | 88.9 | 90.0 | 91.2 | n.t. | 93.0 |
| 17 | 93.2 | 91.5 | 88.0 | 86.7 | n.t. | n.t. | 78.7 | 85.2 |
| 20 | 93.1 | 91.7 | 87.4 | 88.3 | 88.4 | 89.2 | n.t. | 92.7 |

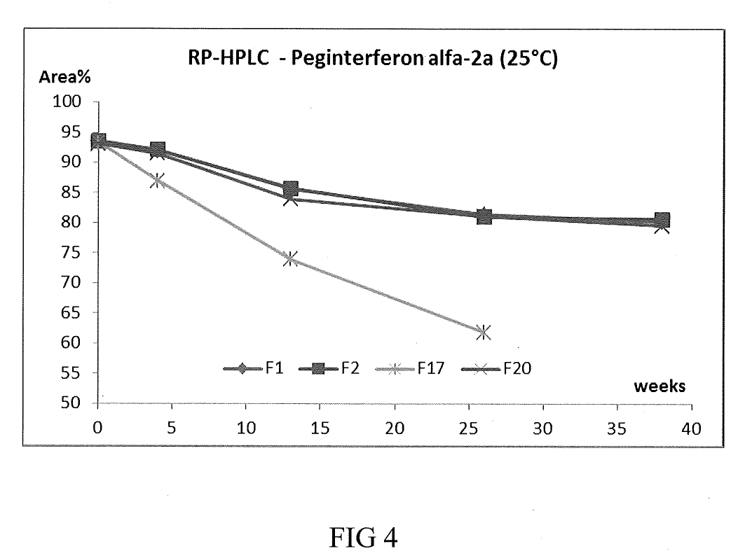

Table 4 below presents data showing the ratio of the purity of pegylated alpha-2α interferon to content of non-oxidized active pharmaceutical ingredient (API), as measured by HPLC, in different formulations over time at 25° C. storage. This data is represented in FIG. 4.

TABLE 4

| Formulation | Initial | 4 weeks | 13 weeks | 26 weeks | 38 weeks |
|---|---|---|---|---|---|
| 1 | 93.5 | 92.0 | 85.6 | 81.4 | 80.3 |
| 2 | 93.6 | 92.1 | 85.7 | 81.0 | 80.6 |
| 17 | 93.2 | 86.9 | 73.9 | 61.9 | n.t. |
| 20 | 93.1 | 91.5 | 83.9 | 81.1 | 79.5 |

What is claimed is:

1. A stable, isotonic, aqueous solution formulation comprising:
(i) 0.1-0.5 mg/mL alpha-type interferon, preferably pegylated alpha-type interferon;
(ii) 20 mM Acetate buffer system to maintain a pH of 6.0±0.5;
(iii) 5-20 mM L-methionine;
(iv) 120-150 mM sodium chloride;
(v) 0.01-0.07 percent by weight of a surfactant effective to stabilize the alpha-type interferon, preferably pegylated alpha-type interferon against loss of its activity and
(vi) an amount of water for injection sufficient to prepare a solution of the above-listed ingredients.

2. The composition of claim 1 wherein the surfactant is either polysorbate 20 or poloxamer 188.

3. The composition of claim 1 wherein the surfactant is polysorbate 20.

4. The composition of claim 1 wherein the pegylated alpha-type interferon is a physiologically active pegylated alpha-type interferon conjugate of formula (I) 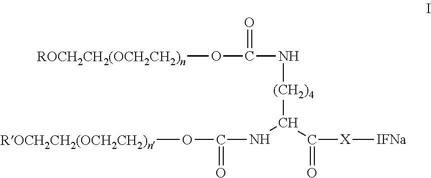
wherein R and R′ are methyl, X is NH, the average sum of n and n′ is 850 to 1000, the molecular weight of the polyethylene glycol units is about 40 kDa and the interferon alpha is an interferon alpha-2a.

5. A stable aqueous solution formulation comprising:
(i) Pegylated alpha-type interferon of formula (I) 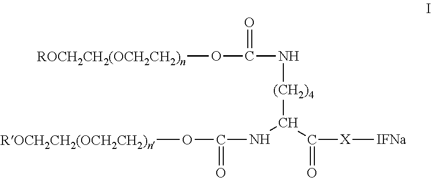
wherein R and R′ are methyl, X is NH, the average sum of n and n′ is 850 to 1000, the molecular weight of the polyethylene glycol units is about 40 kDa and the interferon alpha is an interferon alpha-2a;
(ii) acetate buffer to maintain a pH of 6.0±0.5;
(iii) L-methionine;
(iv) sodium chloride;
(v) Polysorbate 20; and,
(vi) water for injection.

6. The formulation of claim 5, wherein the formulation comprises 0.36 mg/ml pegylated alpha-type interferon, 20 mM acetate buffer, 10 mM L-methionine, 137 mM sodium chloride, 0.02% by weight polysorbate 20, and as much as sufficient of water for injection for 1 ml.

7. A stable aqueous solution formulation comprising:
(i) Pegylated alpha-type interferon of formula (I) 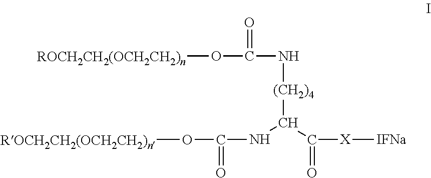
wherein R and R′ are methyl, X is NH, the average sum of n and n′ is 850 to 1000, the molecular weight of the polyethylene glycol units is about 40 kDa and the interferon alpha is an interferon alpha-2a;
(ii) acetate buffer to maintain a pH of 6.0±0.5;
(iii) L-methionine;
(iv) sodium chloride;
(v) poloxamer 188; and,
(vi) water for injection.

8. The formulation of claim 7, wherein the formulation comprises 0.36 mg/ml pegylated alpha-type interferon, 20 mM acetate buffer, 10 mM L-methionine, 137 mM sodium chloride, 0.02% by weight poloxamer 188, and as much as sufficient of water for injection for 1 ml.

9. An article of manufacture comprising a packaging material and the formulation of any one of claims 1-8, wherein the packaging material is a glass vial.

10. An article of manufacture comprising a packaging material and the formulation of any one of claims 1-8, wherein the packaging material is a prefilled syringe.

11. A method for preparing a stable, isotonic, aqueous solution formulation comprising high biological alpha-type interferon activity, preferably pegylated alpha-type interferon activity, comprising admixing:
(i) 0.1-0.5 mg/mL alpha-type interferon, preferably pegylated alpha-type interferon;
(ii) 20 mM Acetate buffer system to maintain a pH of 6.0±0.5;
(iii) 10 mM L-methionine;
(iv) 140 mM sodium chloride;
(v) 0.02 percent by weight of a surfactant effective to stabilize the alpha-type interferon, preferably pegylated alpha-type interferon against loss of its activity; and,
(vi) sufficient amount of water to make an aqueous solution.
